(19) United States
(12) Patent Application Publication
(10) Pub. No.: US 2011/0275870 A1
(43) Pub. Date: Nov. 10, 2011

(54) REMOVABLE BASKETS CONTAINING FILTRATION PARTICLES FOR FIXED BED REACTORS

(75) Inventors: Abdelhakim Koudil, Saint-Laurent-De-Mure (FR); Isabelle Guibard, St Symphorien D'Ozon (FR); Christophe Boyer, Charly (FR); Frederic Bazer-Bachi, Saint Genis-Laval (FR)

(73) Assignee: IFP Energies nouvelles, RUEIL-MALMAISON CEDEX (FR)

(21) Appl. No.: 13/100,898

(22) Filed: May 4, 2011

(30) Foreign Application Priority Data
May 6, 2010 (FR) 10/01.956

(51) Int. Cl. C07C 5/00; B01J 8/00

(52) U.S. Cl. 585/251; 422/187

(57) ABSTRACT

A filtration device placed upstream of a catalytic bed of a reactor functioning in downflow or upflow mode consists of a plurality of baskets containing filtration particles with a principal dimension (dp), said baskets being supported by a base plate fixed to the walls of the reactor, termed the filtration plate, the assembly of baskets covering at least the central portion of the section of the reactor, and forming a filtration bed with height Hp. Application of said filtration device to processes for hydrotreatment, selective hydrogenation or conversion of residues or hydrocarbon cuts with a boiling point higher than 120° C., preferably higher than 250° C.

### FIELD OF THE INVENTION

The present invention relates to the field of fixed bed reactors used in many fields of refining. Examples which may be cited are hydrogenations of various oil cuts with a boiling point higher than 120° C., preferably higher than 250° C.

More particularly, the invention is applicable to processes treating heavy feeds that can possibly suffer from problems of clogging of the catalytic bed. In such a case, the skilled person will know to use, upstream of the catalytic bed per se, one or more filtration beds intended to protect the catalytic bed from clogging. Said filtration beds, after clogging, have to be regularly replaced with fresh filtration beds, the frequency of such changes depending strongly on the process and on the chemical nature of the clogging particles.

The essential aim of the invention is to facilitate and reduce the frequency of these operations for the replacement of such filtration beds.

Its field of application is large and the present invention is compatible with distributor plate systems which have been developed in the field of fixed beds with two-phase flows, particularly in the case of processes employing a gas-liquid down flow mode. Troubles may arise when charging filtration particles onto the distributor plate of a gas-liquid reactor operating in co-current downflow mode.

The layers of filtering particles constituting the bed are relatively thin, generally a few tens of centimetres, and the difficulty with charging homogeneously in regard to the depth of the bed is largely due to the presence of chimneys on the plate, which chimneys are generally spaced 100 mm to 500 mm apaDense charging is also difficult, as the presence of said chimneys will perturb the level of the particle bed in their vicinity.

By using the removable baskets according to the invention, charging the baskets with filtration particles can be carried out outside the reactor, which facilitates and greatly accelerates the operation, which is thus limited to positioning the charged baskets on the filtration plate.

The height of the filtration particles in each basket can be adjusted in a very precise manner, from basket to basket.

During discharge, it may often be the case that because the filtered particles agglomerate with the filtration particles, the filtration bed constitutes a block which can only be fragmented using a pneumatic drill or other equivalent means. That operation is both expensive and risks damaging the chimneys and other constituent elements of the filtration plate.

Using a pneumatic drill, for example, also risks to damage the wall of the reactor and in particular the corrosion protection layer. The reactor is then weakened and subjected to substantial corrosion risks.

The present invention means that the operation for fragmentation of a filtration bed which might have agglomerated can be avoided since the bed is constituted by a plurality of baskets containing the filtration particles such that even if some baskets have agglomerated, it is still possible to remove them easily. Thus, the present invention can also be used to limit the shut down time of the unit.

### EXAMINATION OF THE PRIOR ART

The prior art in the field of fixed bed reactors with a mono-phase gas or liquid flow or with two-phase gas and liquid flow is large. We only examine documents which describe filtration systems known as filtration plates as it is more particularly in the field of heavy feeds containing clogging particles that the present invention finds applications.

A description of this type of filtration plate is to be found in the document FR 2 889 973 which is in the context of reactors with a downflow co-current of a gas phase and a liquid phase. The filtration plate described in that document is located upstream of a catalytic bed and is constituted by a flat base fixed to the walls of the reactor and supporting a series of chimneys for admitting gas to their upper end, for admitting liquid via their lateral orifices, and for evacuating the gas-liquid mixture via their lower end. The plate supports a filtration bed surrounding the chimneys, said bed being constituted by at least one layer of filtration particles.

The cited document makes no mention of problems with handling and cleaning the filtration bed for which the present invention constitutes a solution.

The present invention goes beyond the context of downflow reactors and also concerns upflow reactors.

### BRIEF DESCRIPTION OF THE FIGURES

FIG. 1 represents a cavalier projection of a filtration basket in which the height of the basket Hp and the length of a side Lp are shown;

FIG. 2 provides some examples of possible shapes of the upper section of the baskets;

FIG. 3 is used to illustrate the example and show the elementary baskets (FIG. 3a) and baskets traversed by a central chimney (FIG. 3b).

### BRIEF DESCRIPTION OF THE INVENTION

The present invention can be defined as a device with removable baskets enclosing solid filtration particles, the assembly of baskets allowing the whole of the filtration bed, generally located upstream of the catalytic bed, to be packed.

The term “upstream” should be understood in accordance with the direction of flow of the reaction fluid (or fluids).

In the remainder of the text, the term “filtration bed” is used to denote the assembly of filtration baskets.

More precisely, the present invention consists of a filtration device placed upstream of a catalytic bed of a mono-phase or two-phase reactor functioning in downflow or upflow mode, said device consisting of a plurality of baskets containing filtration particles with a principal dimension (dp), said baskets being supported by a base plate fixed to the walls of the reactor, termed the filtration plate. The assembly of baskets covers in a compact way at least the central portion of the section of the reactor, and forms a filtration bed with a height Hp, each basket being defined by at least three substantially vertical lateral walls and by a lower wall, the lateral walls and the lower wall of the baskets being pierced with orifices with a smaller size than that of the filtration particles (dp). The expression “close packs” means the fact that there are no voids between the baskets which thus form a continuous cover of the central portion of the reactor.

In many applications, and in particular for processes for hydrotreatment, selective hydrogenation or conversion of residues or hydrocarbon cuts having a boiling point of more than 120° C., preferably more than 250° C., the filtration device of the present invention is incorporated into a filtration plate comprising vertical chimneys for mixing the gas and liquid phases. The height Hp of the filtration baskets is thus less than the height of said chimneys by a value in the range 0 to 100 mm.

In general, the filtration baskets are defined by a chimney located at each extremity of the sides of said basket (also termed the angle).

A distinction can be drawn between the filtration baskets having a contact with the wall of the reactor, termed peripheral baskets, and the filtration baskets which have no contact with the wall of the reactor, termed central baskets.

In a variation of the present invention, the central filtration baskets are traversed by one or more central chimneys.

In another variation of the present invention, a portion of the baskets having contact with the wall of the reactor, termed peripheral baskets, is removed and replaced by filtration particles disposed directly in the space separating the central baskets from the wall of the reactor. The filtration particles are then generally introduced in bulk.

Certain adjacent baskets may be bolted, clamped or welded together, as well as to the horizontal surface of the plate, in order to improve the rigidity of the assembly of said baskets.

In another variation of the present invention, the upper layer of the catalytic bed, over a height which is generally in the range 200 mm to 1500 mm, is enclosed in baskets termed catalytic baskets in a manner analogous to that of the filtration baskets.

The application of the filtration device of the present invention more particularly concerns processes for hydrotreatment, selective hydrogenation or conversion of residues or hydrocarbon cuts with a boiling point of more than 120° C., preferably more than 250° C.

### DETAILED DESCRIPTION OF THE INVENTION

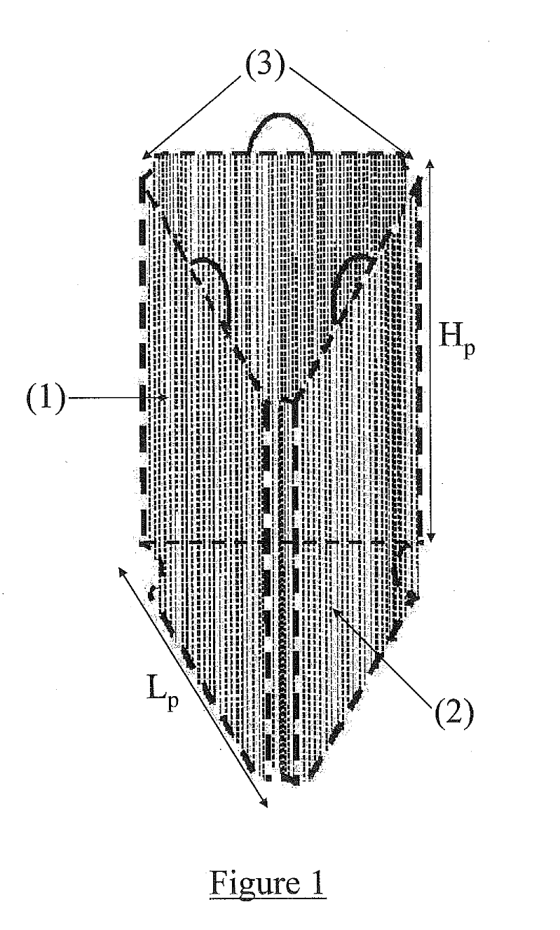

A filtration basket of the present invention as shown in FIG. 1 is constituted by at least three substantially vertical lateral walls 1, and a lower wall 2 produced from a metal screen or a Johnson type screen or from a metal plate pierced with orifices.

The size of the mesh of the screen or the orifices is such that it is strictly smaller than the mean dimension of the solid particles contained in the basket.

The principal dimensions of the basket are the height Hp, and the length Lp of one side, which defines the horizontal section of said basket.

The horizontal section of a basket may be triangular (case of 3 lateral walls), quadrangular (case of 4 lateral walls) or have a curved shape in order to follow the shape of the reactor wall.

Assembly of the baskets is adapted in order to pack the entire section of the reactor, leaving only a free space or functional clearance between the lateral walls of adjacent baskets in order to allow them to be introduced or removed individually, i.e. basket by basket. This is what is meant by the fact that the baskets cover in a compact way at least the central portion of the section of the reactor.

The particles which have contact with the walls of two adjacent baskets are separated by the thickness of said walls, which prevents clogging between the baskets.

The baskets which have one wall in contact with the wall of the reactor have on the corresponding lateral walls, a curvature which matches the curvature of the reactor wall. However, in some cases, these peripheral baskets (Pp) or at least some of them, may be suppressed. The filtration particles are then introduced directly into the space included between the walls of the central baskets (Pc) and the wall of the reactor.

In the case of a reactor with a gas and liquid downflow, the shape of the upper section of a basket must accommodate with the presence of chimneys, these chimneys allowing, depending on the case, the passage of gas or the passage of liquid, and usually the mixed passage of gas and liquid in said chimneys.

Two cases are possible:

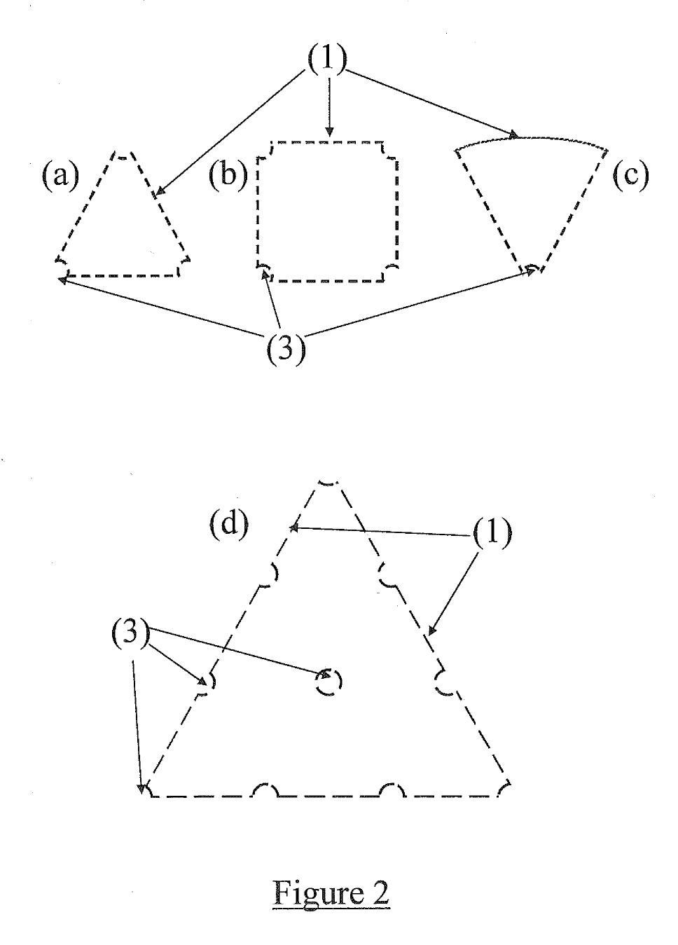

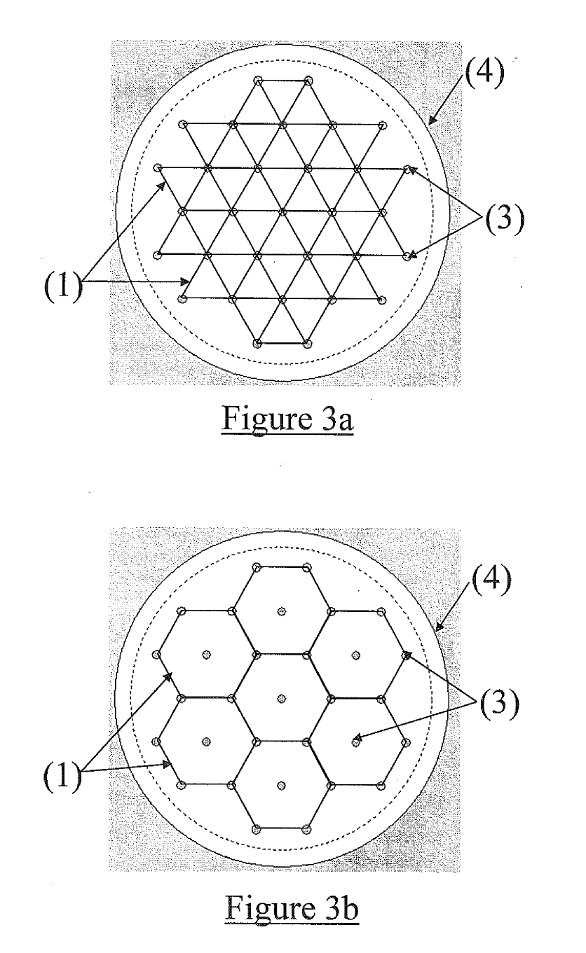

a) a basket with its angles defined by a chimney (3), and the basket does not have any emplacement for the passage of a central chimney In practice, such baskets, termed elementary baskets, are thus disposed between neighbouring chimneys and match the pitch of said chimneys. This is the solution that corresponds to the smallest baskets and thus to the greatest number of baskets to pack the whole section of the reactor. An example of baskets of this type is shown in FIGS. 2a, 2b, 2c and FIG. 3a;
b) a basket with its angles defined by a chimney (3) and which is traversed by at least one central chimney In this case, the basket covers a larger surface area which corresponds to a multiple of the section of the elementary baskets. As an example, FIG. 3b shows a basket with a hexagonal shape with a chimney in the centre and 6 chimneys, one at each of the angles of the hexagon. FIG. 2d shows a basket with a triangular shape traversed by a central chimney, with one chimney at each angle and two chimneys on each side of the triangle.

The height of the baskets, Hp, is generally less than or equal to the height of the chimneys.

The baskets are usually provided with gripping means for handling them by means of a jack ring type system.

The filtration device of the present invention consists of a plurality of baskets containing filtration particles with a principal dimension (dp), said baskets being supported by a base plate fixed to the walls of the reactor, and termed the filtration plate.

Said baskets are generally identical in shape with the possible exception of the baskets having contact with the wall of the reactor which have curved portions of their corresponding walls in order to match the shape of the walls (4) of the reactor.

The assembly of baskets packs at least the central portion of the section of the reactor, and forms a filtration bed with height Hf, each basket being defined by at least three substantially vertical lateral walls defining a triangular or quadrangular section or any other geometric form that can allow the basket to be inserted between the chimneys, and by a lower wall. The lateral and lower walls are pierced with orifices with a size which is smaller than that of the filtration particles (dp).

The baskets which do not have any of their walls in contact with the wall of the reactor are termed the central baskets and can be distinguished from the baskets which have a portion of their walls in contact with the wall of the reactor and which are termed peripheral baskets.

In a variation of the present invention, a portion of the baskets which have contact with the wall of the reactor, termed peripheral baskets, may be suppressed and replaced by filtration particles disposed directly in the space separating the central baskets from the wall of the reactor.

A basket is defined geometrically by the number of its lateral walls, the height Hp of said lateral walls, and by the length Lp of one of its sides.

FIG. 3 shows several possible forms for the section of a basket.

In the case in which the filtration plate supporting the baskets has chimneys, which is generally the case for two-phase flow reactors, the baskets have a section which may be triangular or quadrangular, and the angles of the triangle or quadrilateral correspond to the positioning of the chimneys, as can be seen in FIG. 3.

The baskets termed central baskets may in some cases be traversed by a chimney, thereby increasing the possibilities for the geometric shape. They may be bolted, clamped or welded together and to the horizontal surface of the plate supporting them.

The notion of baskets may also be applied to a fraction of the catalytic bed itself, limiting the height of the catalytic baskets between 200 mm and 1500 mm Clearly, the scope of the present invention includes using both filtration baskets and catalytic baskets.

### Example, in Accordance with the Invention

This example shows two cases of dimensioning of the filtration baskets for a reactor for selective hydrogenation of a cut with a boiling point in the interval 150° C. to 200° C. This reactor was provided with a distributor plate having contact with the walls of the reactor and provided with chimneys to mix gas and liquid.

The principal characteristics of the distributor plate, chimneys and filtration baskets are as follows:
Diameter of catalytic bed: 1 m;
Characteristics of chimneys:
   diameter: 25 mm;
   pitch between chimneys: 150 mm;
   height: 350 mm;
   total number of chimneys: 31;
   the chimneys are perforated with:
   3 holes of 9 mm located at 50 mm from the level of the plate and spacing at 120° to each other;
   3 holes of 9 mm located at 250 mm above the upper level of the catalyst and spacing at 120° to each other.
Height of filtration bed: 270 mm;
Height of filtration baskets: 310 mm.

No baskets have contact with the wall of the reactor. In the space included between the wall of the reactor and the central baskets, bulk filling was carried out using the same particles as in the baskets. Two designs of basket can be defined, both of which satisfying the requirements for packing the reactor.

Each basket is provided with means for easy handling by a crane or any other lifting means.
Design 1: the filtration bed is constituted by elementary baskets, i.e. fitting between the chimneys, i.e. 3+9+9+9+9+3=42 baskets (see FIG. 3);
Design 2: the filtration bed is constituted by baskets traversed by an internal chimney: i.e. 7 baskets, which were hexagonal in shape (see FIG. 3).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding FR application Ser. No. 10/01.956, filed May 6, 2010 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A device for distribution and filtration placed upstream a catalytic bed of a reactor functioning in downflow or upflow mode, consisting of a plurality of baskets containing filtration particles with a principal dimension (dp), said baskets being supported by a plate fixed to the walls of the reactor, termed the filtration plate, the assembly of baskets covering in a compact manner at least the central portion of the section of the reactor and forming a filtration bed with a height Hp, each basket being defined by at least three substantially vertical lateral walls and by a lower wall, the lateral walls and the lower wall of the baskets being pierced with orifices with a smaller size than that of the filtration particles (dp).

2. A distribution and filtration device according to claim 1 in which, when the filtration plate comprises vertical chimneys, the height Hp of the filtration baskets is less than the height of said chimneys by a value in the range 0 to 100 mm.

3. A distribution and filtration device according to claim 2, in which the filtration baskets are defined by a chimney located at each extremity of the sides of said basket.

4. A distribution and filtration device according to claim 2, in which the central filtration baskets are traversed by one or more central chimneys.

5. A distribution and filtration device according to claim 1, in which a portion of the baskets having contact with the wall of the reactor, termed peripheral baskets, is dispensed with and replaced by filtration particles disposed directly in the space separating the central baskets from the wall of the reactor.

6. A distribution and filtration device according to claim 1, in which a portion or all of the adjacent baskets are bolted, clamped or welded together and/or to the horizontal surface of the plate, in order to improve the rigidity of the assembly of said baskets.

7. A distribution and filtration device according to claim 1, in which the filtration baskets are supplemented with baskets having a shape similar to said filtration baskets and which enclose the upper layer of the catalytic bed over a height in the range 200 to 1500 mm.

8. In a method for hydrotreatment, selective hydrogenation or conversion of residues or hydrocarbon cuts with a boiling point of more than 120° C., the improvement comprising subjecting said residues or cuts to a distribution and filtration device according to claim 1
